(19) United States
(12) Patent Application Publication
(10) Pub. No.: US 2008/0249385 A1
(43) Pub. Date: Oct. 9, 2008

(54) ISOLATED INTRAVENOUS ANALYTE MONITORING SYSTEM

(76) Inventor: Luong Ngoc Phan, San Clemente, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/696,675

(22) Filed: Apr. 4, 2007

(51) Int. Cl. A61B 5/05

(52) U.S. Cl. 600/347

(57) ABSTRACT

A continuous intravenous analyte monitoring system includes an amperometric biosensor detecting an analyte concentration in the blood, a controller receiving a signal from the biosensor and computing the concentration, and an isolation device isolating the biosensor from EMI. A CPU may be coupled to the controller via the isolation device for continuous output of sensed concentration to a display unit. Isolated circuits may include a temperature sensor transmitting biosensor temperature to the controller for correction of the computed concentration, a multiplexer combining biosensor and temperature sensor signals, and an A/D converter converting multiplexed input to the controller. The biosensor may be a multi-electrode sensor having a working electrode immobilizing an glucose oxidase enzyme to detect blood glucose concentration. The biosensor and temperature sensor may be located in vivo using a catheter for continuous monitoring.

### BACKGROUND

1. Field of the Invention

The invention relates generally to an intravenous analyte monitoring system. More specifically, the invention relates to an electronic system for electrically isolating an intravenous amperometric biosensor.

2. Description of Rated Art

Controlling blood glucose levels for diabetics and other patients can be a vital component in critical care, particularly in an intensive care unit (ICU), operating room (OR), or emergency room (ER) setting where time and accuracy are essential. Presently, the most reliable way to obtain a highly accurate blood glucose measurement from a patient is by a direct time-point method, which is an invasive method that involves drawing a blood sample and sending it off for laboratory analysis. This is a time-consuming method that is often incapable of producing needed results in a timely manner. Other minimally invasive methods such as subcutaneous methods involve the use of a lancet or pin to pierce the skin to obtain a small sample of blood, which is then smeared on a test strip and analyzed by a glucose meter. While these minimally invasive methods may be effective in determining trends in blood glucose concentration, they do not track glucose accurately enough to be used for intensive insulin therapy, for example, where inaccuracy at conditions of hypoglycemia could pose a very high risk to the patient.

Amperometric biosensors are well known in the medical industry for analyzing blood chemistry. These types of sensors contain enzyme electrodes, which typically include an oxidase enzyme, such as glucose oxidase, that is immobilized behind a membrane on the surface of an electrode. In the presence of blood, the membrane selectively passes an analyte of interest, e.g. glucose, to the oxidase enzyme where it undergoes oxidation or reduction, e.g. the reduction of oxygen to hydrogen peroxide. Amperometric biosensors function by producing an electric current when a potential sufficient to sustain the reaction is applied between two electrodes in the presence of the reactants. For example, in the reaction of glucose and glucose oxidase, the hydrogen peroxide reaction product may be subsequently oxidized by electron transfer to an electrode. The resulting flow of electrical current in the electrode is indicative of the concentration of the analyte of interest.

While amperometric biosensors have been demonstrated in a static laboratory setting, there are many problems impeding the development of these sensors for intravenous use in a critical care setting. One of these problems is noise interference. A patient undergoing critical care is likely to have other monitors and sensors connected in and around the vital organ areas. For example, leads from an imaging device, a blood pressure monitor, an electrocardiograph, or a temperature sensing device may all need to be installed near the chest cavity of the patient. These devices are common sources of electrical, magnetic, or ground noise that can interfere with measurements taken by an amperometric sensor and cause unacceptably inaccurate readings.

With diabetes reaching epidemic proportions in the United States and elsewhere, a genuine need exists for a technology that measures blood glucose concentration quickly, reliably, and frequently, especially for the critically ill.

### SUMMARY

The invention provides a system for continuous intravenous monitoring of blood chemistry using an amperometric biosensor that is electrically isolated from external noise sources. The monitoring system may include a biosensor, a controller, and an isolation device to isolate the biosensor from electromagnetic interference (EMI). The controller may be coupled to the biosensor to receive output from the biosensor and to compute a concentration level of an analyte of interest in the blood. The system may also include a computer or CPU, and the isolation device may be coupled between the controller and the CPU. The CPU provides power for the system, and outputs the computed analyte level to a display. The isolation device provides a signal transmission path between the controller and the CPU, while electrically isolating the controller from the CPU and from the display unit to prevent noise from interfering with the biosensor signal. In one embodiment, the system may be a glucose monitoring system and the analyte of interest may be glucose.

The biosensor may include first and second working electrodes, a reference electrode, and a counter electrode. The first working electrode carries a glucose-sensitive enzyme that reacts with glucose and outputs a signal current proportional to glucose concentration. The second working electrode may be configured without the enzyme, but may otherwise be identical to the first working electrode to allow for correction of signal current from the first working electrode caused by phenomena other than the enzyme. The reference electrode provides a reference voltage for the first and second working electrodes. The counter electrode provides a return path to the blood for the majority of electrons produced from a chemical reaction.

In one embodiment, a continuous glucose monitoring system includes a potentiostat coupled between the biosensor and the controller. The potentiostat may receive signal output from the working electrodes and transfer the output to the controller. The potentiostat may also provide a bias voltage to energize the first and second working electrodes at a fixed potential relative to the reference electrode to sustain a desired chemical reaction. In another embodiment, the system may include a sensor for monitoring patient temperature. The temperature sensor may output signals to the controller for use in correcting a computed analyte level. The biosensor and temperature sensor may be located in vivo using a catheter for continuous monitoring. The isolation device may include a DC/DC converter coupled between the CPU and an isolated portion of the system to provide DC power for the controller and associated electronics. Further, the isolation device may include a spacing barrier to physically separate isolated circuits from non-isolated circuits.

### BRIEF DESCRIPTION OF THE DRAWINGS

The exact nature of this invention, as well as the objects and advantages thereof will become readily apparent from consideration of the following specification in conjunction with the accompanying drawings in which like reference numerals designate like parts throughout the figures thereof and wherein:

FIG. 1 is a schematic diagram of a four-electrode biosensor according to an embodiment of the invention.

FIG. 2 is a circuit diagram of a biosensor and potentiostat in a continuous glucose monitoring system according to an embodiment of the invention.

FIG. 3 is a block diagram of a continuous glucose monitoring system according to an embodiment of the invention.

FIGS. 4A-4D are circuit diagrams of a continuous glucose monitoring system according to an embodiment of the invention.

### DETAILED DESCRIPTION

The present invention provides a system that allows physicians or other health care to continuously monitor a patient's blood chemistry using a specialized sensor that can be installed intravenously. The specialized sensor, or biosensor, may be a miniaturized electrode built into a thin, flexible strip called a flex circuit. The flex circuit can be made small enough to be mounted on a catheter or other medical probe and positioned inside a large blood vessel of a patient. The biosensor electrode may contain an enzyme capable of reacting with a substance in the blood, such as blood glucose, to generate electrical signals. These signals are sent along tiny electrical wires back through the catheter to an electronic box, which calculates the amount of substance in the blood, for example, the blood glucose concentration. The results can then be conveniently displayed for the attending physician. The electronic box may also be specially designed to isolate the biosensor signals from interfering noise and electrical static, so that highly accurate measurements can be taken and displayed. Since the biosensor can operate continually when it is installed in the blood vessel, the results may be seen in real time whenever they are needed. This has the advantage of eliminating costly delays that occur using the old method of extracting blood samples and sending them off for laboratory analysis. In addition, use of the intravenous biosensor means that the patient does not suffer any discomfort from periodic blood drawing, or experience any blood loss whenever a measurement needs to be taken.

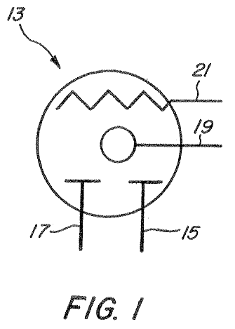

FIG. 1 is a schematic diagram of a four-electrode biosensor 13 according to an embodiment of the invention. In one embodiment, the biosensor 13 may be a miniaturized electrode mounted on a flex circuit formed on a substrate such as polyimide. The flex circuit may have a length between about 1.00 inch and about 3.00 inches and a width between about 0.20 inches and about 0.40 inches. For intravenous monitoring, this size flex circuit may be affixed to a catheter such as a central venous catheter (CVC), a peripherally inserted central catheter (PICC), or other commonly used peripheral intravenous (IV) catheters.

The biosensor 13 may include two working electrodes: a first working electrode 15 and a second working electrode 17. The first working electrode 15 may be a platinum based enzyme electrode, i.e. an electrode containing or immobilizing an enzyme layer. In one embodiment, the first working electrode 15 may immobilize an oxidase enzyme, such as in the sensor disclosed in U.S. Pat. No. 5,352,348. In another embodiment, the biosensor 13 may be a glucose sensor, in which case the first working electrode 15 may immobilize a glucose oxidase enzyme. The first working electrode 15 may be formed using platinum, or a combination of platinum and graphite materials. Other embodiments are possible in which the first working electrode 15 may be formed from other conductive materials. The second working electrode 17 may be identical in all respects to the first working electrode 15, except that it may not contain an enzyme layer.

The biosensor 13 may further include a reference electrode 19 and a counter electrode 21. The reference electrode 19 may establish a fixed potential from which the potential of the counter electrode 21 and the working electrodes 15 and 17 may be established. In one embodiment, the reference electrode 19 may be a silver/silver chloride type deposited or formed on a flex circuit substrate. In this case, the reference potential may be Nernstian. For the silver/silver chloride reference electrode 19, the reference potential is maintained by the following half-reaction:

Ag⁰→Ag⁺+e⁻

In another embodiment, the reference electrode 19 may be made from any suitable conductive material, and may have its reference potential established by an externally located potentiostat.

The counter electrode 21 may be constructed from conductive materials similar to those used for forming the working electrodes 15 and 17, such as platinum or graphite. The counter electrode 21 may provide a working area for conducting the majority of electrons produced from the oxidation chemistry back to the blood solution. Otherwise, excessive current may pass through the reference electrode 19 and reduce its service life. In one embodiment, the counter electrode 21 may be formed with a surface area greater than that of the working electrode 15 or the working electrode 17.

In one embodiment, the biosensor 13 may be formed by applying one or more working electrodes 15 and 17, reference electrode 19, and counter electrode 21 to a flex circuit substrate using a thick film process and inks. The electrode materials (e.g., platinum, silver, and/or graphite) may be formulated as an ink for application to the substrate using a thick film process and cured accordingly.

The biosensor 13 may operate according to an amperometric measurement principle, where the working electrode 15 is held at a positive potential relative to the reference electrode 19. In one embodiment of a glucose monitoring system, the positive potential is sufficient to sustain an oxidation reaction of hydrogen peroxide, which is the result of glucose reaction with glucose oxidase. Thus, the working electrode 15 may function as an anode, collecting electrons produced at its surface that result from the oxidation reaction. The collected electrons flow into the working electrode 15 as an electrical current. In one embodiment with the working electrode 15 coated with glucose oxidase, the oxidation of glucose produces a hydrogen peroxide molecule for every molecule of glucose when the working electrode 15 is held at a potential between about +450 mV and about +650 mV. The hydrogen peroxide produced oxidizes at the surface of the working electrode 15 according to the equation:

H₂O₂→2H⁺+O₂+2e⁻

The equation indicates that two electrons are produced for every hydrogen peroxide molecule oxidized. Thus, under certain conditions, the amount of electrical current may be proportional to the hydrogen peroxide concentration. Since one hydrogen peroxide molecule is produced for every glucose molecule oxidized at the working electrode 15, a linear relationship exists between the blood glucose concentration and the resulting electrical current. The embodiment described above demonstrates how the working electrode 15 may operate by promoting anodic oxidation of hydrogen peroxide at its surface. Other embodiments are possible, however, wherein the working electrode 15 may be held at a negative potential. In this case, the electrical current produced at the working electrode 15 may result from the reduction of oxygen. The following article provides additional information on electronic sensing theory for amperometric glucose biosensors: J. Wang, “Glucose Biosensors: 40 Years of Advances and Challenges,” Electroanaylsis, Vol. 13, No. 12, pp. 983-988 (2001).

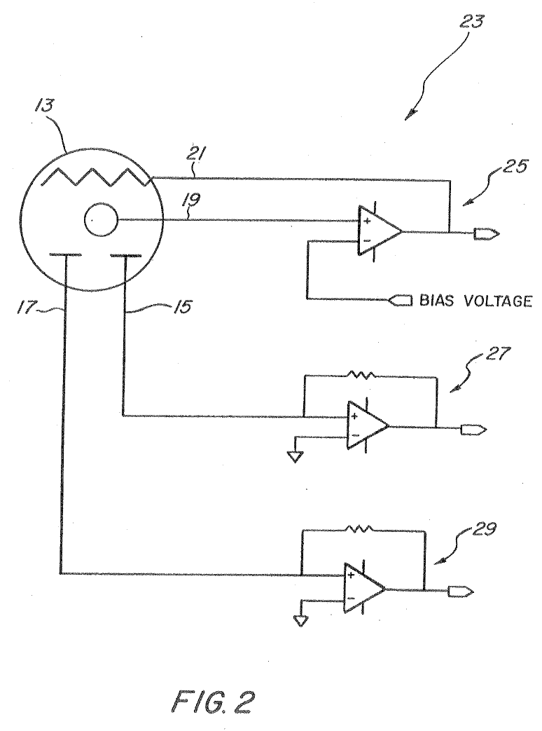

FIG. 2 is a circuit diagram of a portion of a continuous glucose monitoring system 23 according to an embodiment of the invention. FIG. 2 shows the biosensor 13 coupled to an amplification stage of a potentiostat 33. The potentiostat 33 performs several functions. The first of these is maintaining a desired voltage at the working electrodes 15 and 17 with respect to the reference potential established by the reference electrode 19. The voltage level provided to the working electrodes 15 and 17 may be selected to sustain a desired chemical reaction on the working electrodes 15 and 17. In one embodiment, the voltage level for each working electrode 15 and 17 is established between about −450 mV and about +650 mV with respect to the reference electrode 19. Another function of the potentiostat is receiving electrical current signals from the working electrodes 15 and 17 for output to a controller, As the potentiostat 33 works to maintain a constant voltage for the working electrodes 15 and 17, current flow through the working electrodes 15 and 17 may change. The current signals indicate the presence of an analyte of interest in blood. In addition, the potentiostat 33 holds the counter electrode 21 at a voltage level with respect to the reference electrode 19 to provide a return path for the electrical current to the bloodstream, such that the returning current balances the sum of currents drawn in the working electrodes 15 and 17.

To affect these functions, the potentiostat 33 may include three operational amplifiers 25, 27 and 29, configured generally as shown. The operational amplifiers 25, 27 and 29 may be a low input bias current operational amplifier, such as type OPA129UB manufactured by Texas Instruments, Inc. The potentiostat 33 may be located externally from the biosensor 13 and may be coupled thereto via electrical wires running through a catheter or other sensor installation device. When the biosensor 13 is located in a suitable intravenous location, the continuous glucose monitoring system 23 may measure current from the working electrodes 15 and 17 and deliver a usable signal to an output terminal. In another embodiment, the continuous glucose monitoring system 23 may be bipolar to allow operation regardless of whether the current flows to or from the working electrodes 15 and 17.

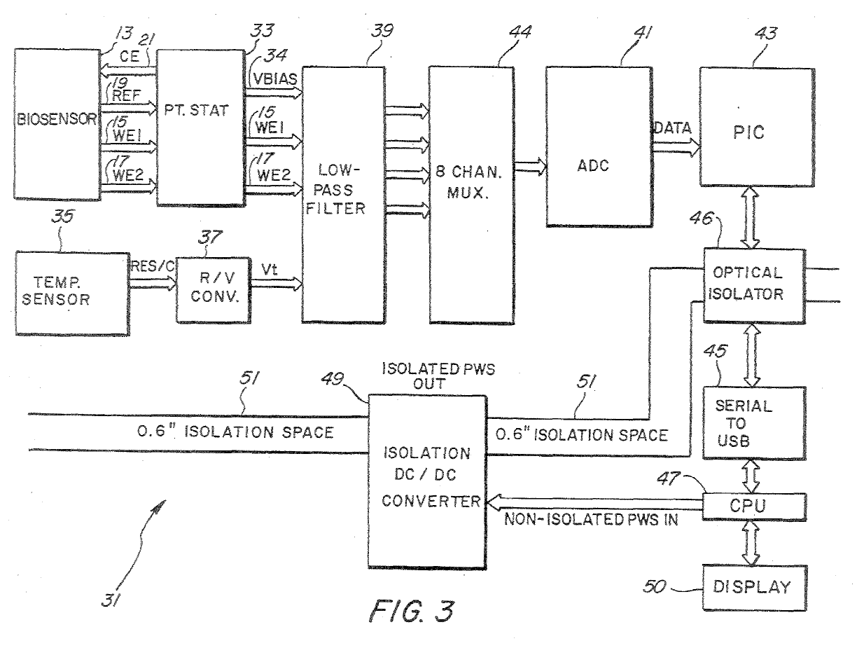

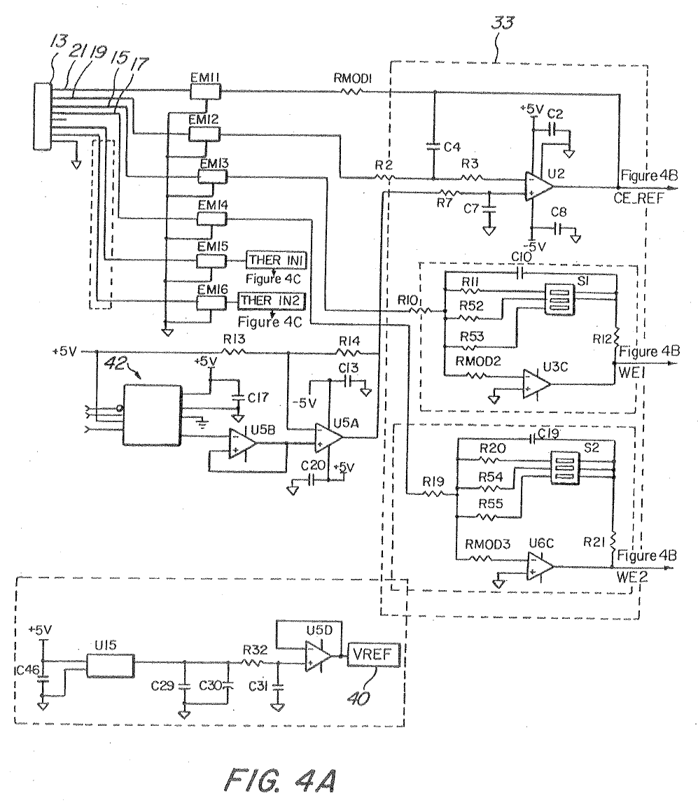

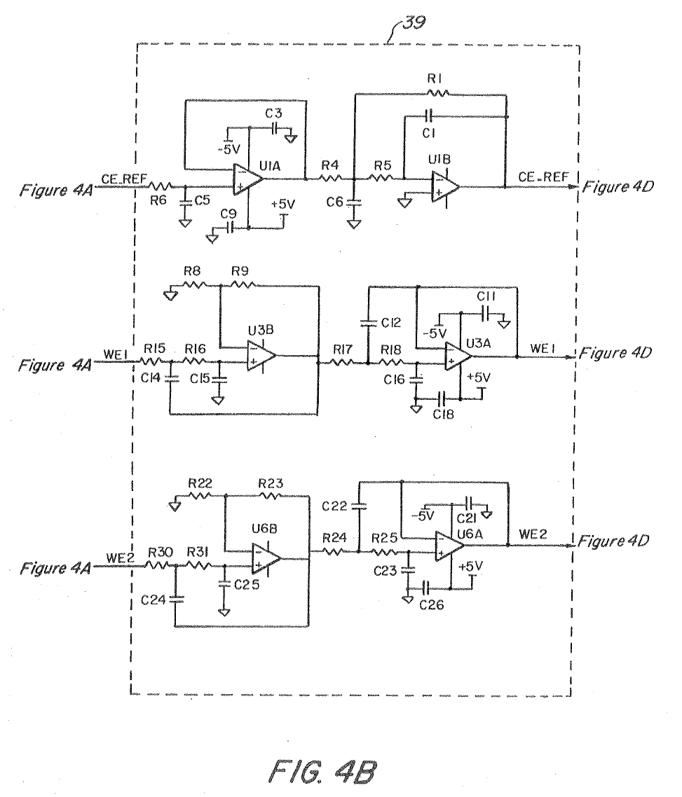

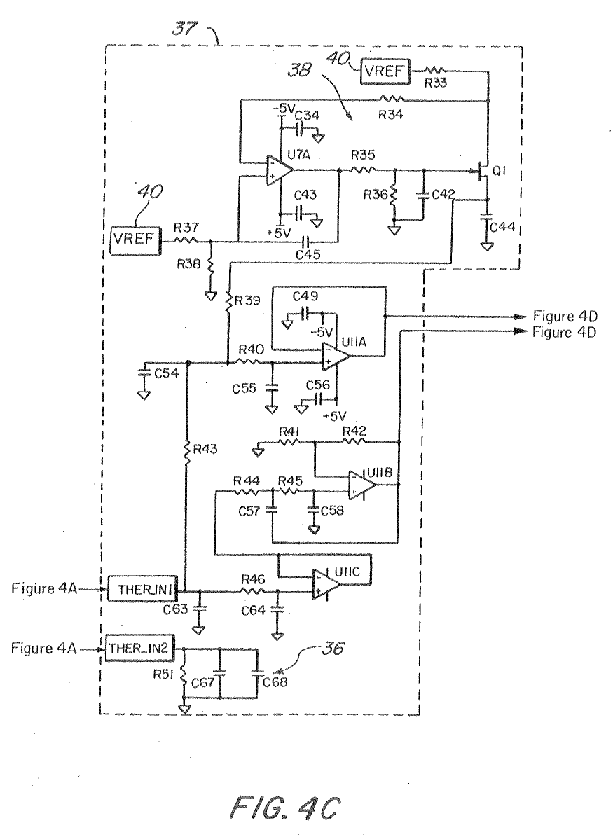

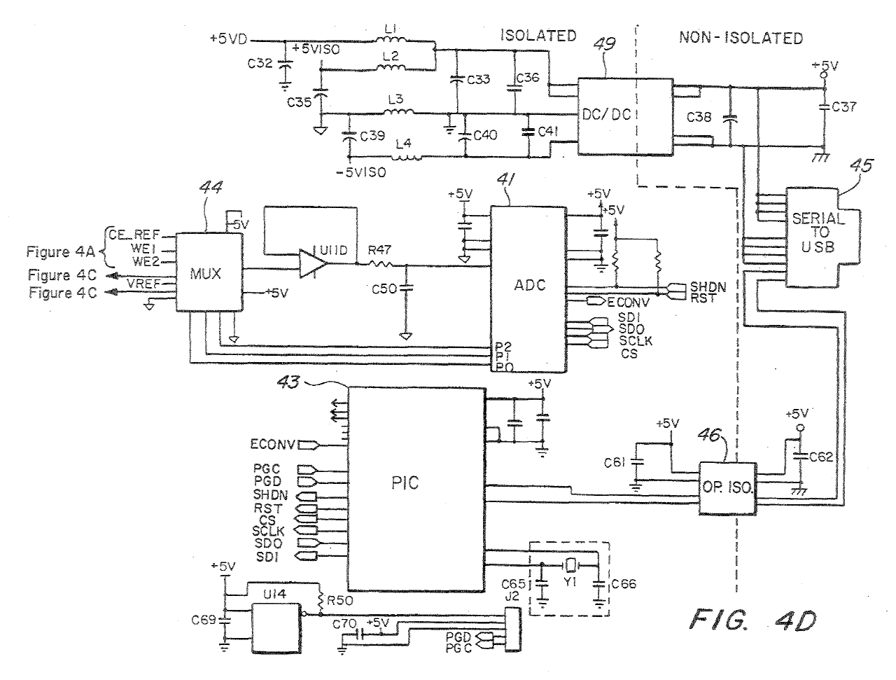

FIG. 3 is a block diagram of a continuous glucose monitoring system 31 according to an embodiment of the invention. In this embodiment, the continuous glucose monitoring system 31 may include the four-electrode biosensor 13, the potentiostat 33, a temperature sensor 35, a resistance-to-voltage (R/V) converter 37, a low-pass filter 39, a multiplexer 44, an analog-to-digital converter (ADC) 41, a peripheral interface controller (PIC) 43, an optical isolator 46, a serial-to-USB converter 45, a processor or CPU 47, an isolation DC/DC converter 49, and a display unit 50. FIGS. 4A, 4B, 4C and 4D are circuit diagrams of the continuous glucose monitoring system 31 according to an embodiment of the invention.

The potentiostat 33 tracks the potential REF for the reference electrode 19, and maintains a constant voltage between the reference electrode 19 and the working electrodes 15 and 17. The potentiostat 33 receives output signal WE1 from the working electrode 15 and output signal WE2 from the working electrode 17. After conditioning these signals, the potentiostat 33 may then output WE1 and WE2 to the low-pass filter 39. The potentiostat 33 may also output to the low-pass filter 39 the voltage potential VBIAS 34 between the counter electrode 21 and the reference electrode 19.

With reference to FIG. 4A, the biosensor 13 is shown in the upper left, coupled to the potentiostat 33 via inputs EM11 through EM16. The signal lines to inputs EM11, EM12, EM13 and EM14 connect to the counter electrode 21, the reference electrode 19, the working electrode 15, and the working electrode 17, respectively as shown, The signal line to input EM15 connects to a first output from a thermistor 35, and the signal line to input EM16 connects to a second output from the thermistor 35. For convenience, the thermistor 35 outputs are shown originating from a sensor block 13, which in this figure represents a local connection point. For example, the thermistor 35 may be integrated with or installed adjacent to the biosensor 13 in an intravenous catheter, in which case it may be convenient to terminate the thermistor 35 and sensor leads at the same connector. In another embodiment, the thermistor 35 and sensor leads may be terminated at separate locations.

The potentiostat 33 may include a control amplifier U2, such as an OPA129 by Texas Instruments, Inc., for sensing voltage at reference electrode 19 through input EM12. The control amplifier U2 may have low noise (about 15 nV/sqrt(Hz) at 10 kHz), an offset (about 5 μV max), an offset drift (about 0.04 μV max) and a low input bias current (about 20 fA max). The control amplifier U2 may provide electrical current to the counter electrode 21 to balance the current drawn by the working electrodes 15 and 17. The inverting input of the control amplifier U2 may be connected to the reference electrode 19 and preferably may not draw any significant current from the reference electrode 19. In one embodiment, the counter electrode 21 may be held at a potential of between about −600 mV and about −800 mV with respect to the reference electrode 19. The control amplifier U2 should preferably output enough voltage swing to drive the counter electrode 21 to the desired potential and pass current demanded by the biosensor 13. The potentiostat 33 may rely on R2, R3 and C4 for circuit stability and noise reduction, although for certain operational amplifiers, the capacitor C4 may not be needed. A resistor RMOD1 may be coupled between the counter electrode 21 and the output of the control amplifier U2 for division of return current through the counter electrode 21.

The potentiostat 33 may further include two current-to-voltage (I/V) measuring circuits for transmission and control of the output signals from the working electrode 15 and the working electrode 17, through inputs EM12 and EM13, respectively. Each I/V measuring circuit operates similarly, and may include a single stage operational amplifier U3C or U6C, such as a type TLC2264. The operational amplifier U3C or U6C may be employed in a transimpedance configuration. In the U3C measuring circuit, the current sensed by the working electrode 15 is reflected across the feedback resistors R11, R52 and R53. In the U6C measuring circuit, the current sensed in the working electrode 17 is reflected across the feedback resistors R20, R54 and R55. The operational amplifier U3C or U6C may generate an output voltage relative to virtual ground. The input offset voltage of the operational amplifier U3C or U6C adds to the sensor bias voltage, such that the input offset of the operational amplifier U3C or U6C may be kept to a minimum.

The I/V measuring circuits for the working electrode 15 and the working electrode 17 may also use load resistors R10 and R19 in series with the inverting inputs of operational amplifiers U3C and U6C, respectively. The resistance of the load resistors R10 and R19 may be selected to achieve a compromise between response time and noise rejection. Since the I/V measuring circuit affects both the rms noise and the response time, the response time increases linearly with an increasing value of the load resistors R10 and R19, while noise decreases rapidly with increasing resistance. In one embodiment, each of load resistors R10 and R19 may have a resistance of about 100 ohms. In addition to the load resistors R10 and R19, the I/V amplifiers may also include capacitors C10 and C19 to reduce high frequency noise.

In addition, the I/V amplifiers of the potentiostat 33 may each include a Dual In-line Package (DIP) switch S1 or S2. Each DIP switch S1 and S2 may have hardware programmable gain selection. Switches S1 and S2 may be used to scale the input current from the working electrode 15 and the working electrode 17, respectively. For operational amplifier U3C, the gain is a function of RMOD2 and a selected parallel combination of one or more resistors R11, R52 and R53. For operational amplifier U6C, the gain is a function of RMOD3 and a selected parallel combination of one or more resistors R20, R54 and R55. Table 1 below illustrates exemplary voltage gains achievable using different configurations of switches S1 and S2.

TABLE 1
Exemplary Voltage Gain

| | | | I/V Output (U3C, | Voltage at A/D | |
|---|---|---|---|---|---|
| Switch Position (S1 and S2) | | | U6C) V per nA | Input | |
| OPEN | OPEN | OPEN | +4.9 V | +4.9 | V |
| OPEN | OPEN | CLOSED | 10 mV (1–20 nA | 200 | mV |
| | | | Scale) | | |
| OPEN | CLOSED | OPEN | 6.65 mV (1–30 nA | 133 | mV |
| | | | Scale) | | |
| CLOSED | OPEN | OPEN | 5 mV (1–40 nA | 100 | mV |
| | | | Scale) | | |

As shown from Table 1, three gain scale settings may be achieved, in addition to the full scale setting. These settings may be selected to correspond to input ratings at the ADC 41.

The potentiostat 33, or a circuit coupled to the potentiostat 33, may further include a digital-to-analog converter (DAC) 42 that enables a programmer to select, via digital input, a bias voltage V_{BIAS} between the reference electrode 19 and the counter electrode 21. The analog output from the DAC 42 may be cascaded through a buffering amplifier U5B and provided to the non-inverting input of the amplifier U5A. In one embodiment, the amplifier U5A may be a type TLC2264 operational amplifier. The output of the amplifier USA may be bipolar, between ±5 VDC, to establish the programmable bias voltage V_{BIAS} for the biosensor 13. The bias voltage V_{BIAS} is the voltage between the counter electrode 21 and the reference electrode 19. Resistors R13 and R14 may be selected to establish a desired gain for the amplifier U5A and the capacitors C13, C17 and C20 may be selected for noise filtration.

The potentiostat 33, or a circuit coupled to the potentiostat 33, may also establish a reference voltage 40 (VREF) for use elsewhere in the control circuits of the continuous glucose monitoring system 31. In one embodiment, the VREF 40 may be established using a voltage reference device U15, which may be an integrated circuit such as an Analog Devices type AD580M. In another embodiment, the reference voltage 40 may be established at about +2.5 VDC. The reference voltage 40 may be buffered and filtered by an amplifier U5D in combination with resistors and capacitors R32, C29, C30 and C31. In one embodiment, the amplifier U5D may be a type TLC2264 device.

With reference now to FIG. 4B, the low-pass filter 39 is now described. The low-pass filter 39 may provide a two-stage amplifier circuit for each signal CE-REF, WE1 and WE2 received from the potentiostat 33, In one embodiment, a 1 Hz Bessel multi-pole low-pass filter may be provided for each signal. For example, the output signal CE_REF of amplifier U2 may be cascaded with a first stage amplifier U1A and a second stage amplifier U1B. The amplifier U1A, in combination with resistor R6 and capacitor C5, may provide one or more poles. One or more additional poles may be formed using an amplifier U1B in combination with R1, R4, R5, C1 and C6. Capacitors such as C3 and C9 may be added, as necessary, for filtering noise from the +/−5 VDC power supply. Similar low-pass filters may be provided for signals WE1 and WE2. For example, the amplifier U3B may be cascaded with an amplifier U3A to filter WE1. The amplifier U3B in combination with components such as R8, R9, R15, R16, C14 and C15 may provide one or more poles, and the amplifier U3A in combination with components such as R17, R18, C11, C12, C16 and C18 may provide one or more additional poles. Similarly, the amplifier U6B may be cascaded with an amplifier U6A to filter WE2. The amplifier U6B in combination with components such as R22, R23, R30, R31, C24 and C25 may provide a first pole, and the amplifier U6A in combination with components such as R24, R25, C21, C22 and C23 may provide one or more additional poles. Additional similar filters (not shown) may be added for filtering signal Vt received from the R/V converter 37. After the low-pass filter 39 filters out high-frequency noise, it may pass signals CE_REF, WE1 and WE2 to a multiplexer 44.

With reference to FIG. 4C, a temperature sensing circuit including the temperature sensor 35 and the R/V converter 37 is now described. The R/V converter 37 receives input from the temperature sensor 35 at terminals THER_IN1 and THER_IN2. These two terminals correspond respectively to the inputs EM15 and EM16 of FIG. 4A that are connected across the temperature sensor 35. In one embodiment, the temperature sensor 35 may be a thermocouple. In another embodiments the temperature sensor 35 may be a device such as a thermistor or a resistance temperature detector (RTD), which has a temperature dependent resistance. Hereinafter, for purposes of illustration only, the continuous glucose monitoring system 31 will be described that employs a thermistor as the temperature sensor 35.

Since chemical reaction rates (including the rate of glucose oxidation) are typically affected by temperature, the temperature sensor 35 may be used to monitor the temperature in the same environment where the working electrodes 15 and 17 are located. In one embodiment, the continuous glucose monitoring system 31 may operate over a temperature range of between about 15° C. and about 45° C. For continuous monitoring in an intravenous application, the operating temperature range is expected to be within a few degrees of normal body temperature. A thermistor 35 should therefore be selected that may operate within such a desired range, and that may be sized for installation in close proximity to the biosensor 13. In one embodiment, the thermistor 35 may be installed in the same probe or catheter bearing the biosensor 13.

The thermistor 35 may be isolated to prevent interference from other sensors or devices that can affect its temperature reading. As shown in FIG. 4C, the isolation of the thermistor 35 may be accomplished by including in the R/V converter 37 a low-pass filter 36 at input THER_IN2. In one embodiment, the low-pass filter 36 may include a simple R-C circuit coupling input THER_IN2 to signal ground. For example, the filter 36 may be formed by a resistor R51 in parallel with a capacitance, e.g. capacitors C67 and C68.

With the thermistor 35 installed in an intravenous location, its resistance changes as the body temperature of the patient changes. The R/V converter 37 may be provided to convert this change in resistance to the voltage signal Vt. Thus, the voltage signal Vt represents the temperature of the biosensor 13. The voltage signal Vt may then be output to the low-pass filter 39 and used for temperature compensation elsewhere in the continuous glucose monitoring system 31.

In one embodiment, the thermistor 35 may be selected having the following specifications:

$${R}_{th} = {R}_{o} {e}^{β [ \frac{1}{T} - \frac{1}{{T}_{o}} ]} \qquad ( 1 )$$

where,

Rₜₕ is the thermistor resistance at a temperature T;

Rₒ is the thermistor resistance at temperature Tₒ;

β=3500° K+/−5%;

Tₒ=310.15° K; and

T is the blood temperature in K.

The reference resistance Rₛ is selected to yield:

$$\frac{{R}_{th}}{{R}_{s}} = 1.4308 + / - 0.010507 \qquad ( 2 )$$

To determine the blood temperature of a patient, equation (1) may be rewritten as:

$$T = {T}_{o} \frac{β}{{T}_{o} ln ( \frac{{R}_{th}}{{R}_{o}} ) + β} \qquad ( 3 )$$

To compensate the output from the biosensor 13 according to temperature, the resistance R₀ of the thermistor 35 may be converted into a voltage signal Vt. To accomplish this, the R/V converter 37 may provide a current source 38 for running a fixed current through the thermistor 35, One embodiment of a circuit for the current source 38 is shown at the top of FIG. 4C, and includes device Q1 and all components to the right of Q1.

In one embodiment, the current source 38 may provide a desired current through Q1. In one embodiment, the source current through Q1 may be between about 5 μA and about 15 μA. Q1 may be a JFET such as a type SST201. To control the JFET, the output of an operational amplifier U7A may be provided to drive the gate of Q1. The voltage VREF may be divided, as necessary, to place a voltage of about +2VDC at the non-inverting input of the amplifier U7A. For example, a voltage divider may be formed by the resistors R37 and R38 between VREF and the amplifier U7A. The amplifier U7A may be configured as an integrator, as shown, by including a capacitor C45 in a feedback path between the output and the non-inverting input, and the resistor R34 in a feedback path from the drain of Q1 to the inverting input, to maintain the drain voltage of Q1 at about +2V. Components such as R36, C34, C42, C43 and C44 may be included, as desired, for filtration and stability.

The resistor R33 placed between the drain of Q1 and the +2.5V VREF may be selected to establish the source current of Q1 at a desired value, In one embodiment, the source current may be maintained at about 9.8 μA for compliance with a medical device standard such as IEC 60601-1. In one embodiment, the thermistor 35 is classified under that standard as a Type CF device (i.e. a device that comes into physical contact with the human heart), and has limits for electrical current leakage that are set at 10 μA for normal operating conditions, and that are set at 50 μA for a single fault condition The selection of resistor R33 and other components that make up the current source 38 may therefore depend on the desired end use application of the continuous glucose monitoring system 31.

One or more voltage signals Vt may be derived from the thermistor 35 by placing one or more reference resistors R39 and R43 in series with the thermistor 35 to carry the source current of Q1. The voltage signals created by the flow of the source current of Q1 through this series resistance may be filtered for electromagnetic interference (EMI) using capacitors C54 and C63. The voltage signals may be further filtered with passive signal poles formed by R40 and C55, and by R46 and C64. In one embodiment, these poles may be established to provide a crossover frequency at approximately 30 Hz. These passive filters protect amplifiers U11A, U11B and U11C from electrostatic discharge (ESD).

In one embodiment, the amplifiers U11A, U11B and U11C may be type TLC2264 devices selected for low noise (12 nV/sqrtHz at frequency=1 Hz), an offset of about 5 uV max, an offset drift of about 0.04 μV max, and an input bias current of about 1 pA max. The amplifier U11A may form a lowpass filter, and transmit a thermistor reference voltage Vt1 at resistor R43. The amplifier U11B may also form a low-pass filter, and transmit a thermistor input voltage Vt2 at the thermistor 35 that represents a sensed temperature. In one embodiment, the amplifier U11A or U11B may function as a two-pole Butterworth filter having a −3 dB point at about 5.0 Hz+/−0.6 Hz for anti-aliasing. Components such as R41, R42, R44, R45, C49, C56, C57 and C58 may be configured for this purpose. The amplifier U11C may be provided as a buffer amplifier at the input of the amplifier U11B.

The first and second voltage signals Vt output from the R/V converter 37 may then be received by the low-pass filter 39 for additional conditioning. In one embodiment, the low-pass filter 39 may provide a four-pole 5 Hz Butterworth filter for signals Vt. The Butterworth filters may double as anti-aliasing filters to create the four-pole response with a −3 dB point at about 5.0 Hz, and have a gain of about 20 (i.e. 26 dB) to provide an output from about 100 mV to about 200 mV per 1.0 nA.

The signals from the biosensor 13 and the thermistor 35 filtered by the low-pass filter 39 may then be output to the multiplexer 44. As shown in FIG. 4D, the multiplexer 44 may receive the signals CE_REF, WE1, WE2, VREF, and the two Vt signals (Vt1 and Vt2), and combine them into a single signal for transmission to the ADC 41. A buffer amplifier U11 may be provided in this transmission path, along with filtering components such as R47 and C50.

In one embodiment, the multiplexer 44 may be an 8-channel analog multiplexer, such as a Maxim monolithic CMOS type DG508A. The channel selection may be controlled by the PIC controller 43 via the output bits P0, P1 and P2 of the ADC 41. Table 2 illustrates an exemplary channel selection for the multiplexer 44.

The ADC 41 converts analog signals to discrete digital data. The ADC 41 may have n output bits (e.g. P0-P2) used for selecting analog input signals at a 2ⁿ-channel multiplexer 44. In one embodiment, the ADC 41 may be a Maxim type MAXI1133BCAP device having a bipolar input with 16 bits successive approximation, single +5V DC power supply and low power rating of about 40 mW at 200 kSPS. The ADC 41 may have an internal 4.096 V_{REF}, which can be used as a buffer. The ADC 41 may be compatible with Serial Peripheral Interface (SPI), Queued Serial Peripheral Interface (QSPI), Microwire or other serial data link. In one embodiment, the ADC 41 may have the following input channels: bias voltage output (CE_REF), working electrode (WE1), working electrode (WE2), DAC converter voltage (DAC_BIAS), thermistor reference voltage (Vt1), thermistor input voltage (Vt2), reference voltage (2.5VREF), and analog ground (ISOGND).

TABLE 2
Exemplary Channel Selection for the Multiplexer

| P2 | P1 | P0 | Mux. Channel | Analog Inputs Description |
|---|---|---|---|---|
| 0 | 0 | 0 | 0 | Reference electrode 19 control voltage |
| 0 | 0 | 1 | 1 | Working Electrode 15 current to voltage |
| 0 | 1 | 0 | 2 | Working electrode 17 current to voltage |
| 0 | 1 | 1 | 3 | Control & Reference bias voltage |
| 1 | 0 | 0 | 4 | Thermistor Reference voltage Vt1 |
| 1 | 0 | 1 | 5 | Thermistor Input voltage Vt2 |
| 1 | 1 | 0 | 6 | 2.5 V_{REF} voltage |
| 1 | 1 | 1 | 7 | ISOGND voltage |

The digital data from the ADC 41 may be transmitted to the PIC controller 43. The PIC controller 43 may be a programmable microprocessor or microcontroller capable of downloading and executing the software for accurate calculation of analyte levels sensed by the biosensor 13. The PIC controller 43 may be configured to receive the digital data and, by running one or more algorithms contained in integral memory, may compute the analyte (e.g. glucose) level in the blood based on one or more digital signals representing CE_REF, WE1, WE2, DAC_BIAS and 2.5VREF, The PIC controller 43 may also run a temperature correction algorithm based on one or more of the foregoing digital signals and/or digital signal Vt1 and/or Vt2. The PIC controller 43 may derive a temperature-corrected value for the analyte level based on the results of the temperature correction algorithm. In one embodiment, the PIC controller 43 may be a Microchip Technology type PIC18F2520 28-pin enhanced flash microcontroller, with 10-bit A/D and nano-Watt technology, 32 k×8 flash memory, 1536 bytes of SRAM data memory, and 256 bytes of EEPROM.

The input clock to the PIC controller 43 may be provided by a crystal oscillator Y1 coupled to the clock input pins. In one embodiment, the oscillator Y1 may be a CTS Corp. oscillator rated at 4 MHz, 0.005% or +/−50 ppm. Y1 may be filtered using the capacitors C65 and C66. The PIC controller 43 may further include an open drain output U14, for example, a Maxim type MAX6328UR device configured with a pull-up resistor R50 that provides system power up RESET input to the PIC controller 43. In one embodiment, the pull-up resistor R50 may have a value of about 10 kΩ. The capacitors C69 and C70 may be sized appropriately for noise reduction.

In one embodiment, data transfer between the PIC controller 43 and the ADC 41 may be enabled via pins SHDN, RST, ECONV, SDI, SDO, SCLK and CS, as shown. An electrical connector J2, such as an ICP model 5-pin connector, may be used to couple pins PGD and PGC of the PIC controller 43 to drain output U14. The connector J2 may provide a path for downloading desired software into the integral memory, e.g. flash memory, of the PIC controller 43.

The PIC controller 43 may output its results to the CPU 47 via the optical isolator 46 and the serial-to-USB port 45. The optical isolator 46 may use a short optical transmission path to transfer data signals between the PIC controller 43 and the serial-to-USB converter 45, while keeping them electrically isolated. In one embodiment, the optical isolator 46 may be an Analog Devices model ADuM1201 dual channel digital isolator. The optical isolator 46 may include high speed CMOS and monolithic transformer technology for providing enhanced performance characteristics. The optical isolator 46 may provide an isolation of up to 6000 VDC for serial communication between the PIC controller 43 and the serial-to-USB converter 45. The filter capacitors C61 and C62 may be added for additional noise reduction at the +5VDC inputs. At the capacitor C61, the +5VDC power may be provided by an isolated output from the DC/DC converter 49. At the capacitor C62, the +5VDC power may be provided from a USB interface via the CPU 47. In addition to these features, an isolation space 51 may be established (e.g., on a circuit board containing the isolated electrical components) between about 0.3 inches and about 1.0 inches to provide physical separation to electrically and magnetically isolate circuit components on the “isolated” side of the optical isolator 46 from circuit components on the “non-isolated” side. The components segregated onto “isolated” and “non-isolated” sides are indicated by the isolation space 51 on FIG. 3, and by the dashed line on FIG. 4D. In one embodiment, the isolation space may be 0.6 inches.

Generally, an isolation device or isolation means prevents noise from outside the isolated side of the circuit from interfering with signals sensed or processed within the isolated side of the circuit. The noise may include any type of electrical, magnetic, radio frequency, or ground noise that may be induced or transmitted in the isolated side of the circuit. In one embodiment, the isolation device provides EMI isolation between the isolated sensing circuit used for sensing and signal processing, and the non-isolated computer circuit used for power supply and display. The isolation device may include one or more optical isolators 46, DC/DC converters 49, isolation spaces 51, and one or more of the many electronic filters or grounding schemes used throughout the continuous glucose monitoring system 31.

The serial-to-USB converter 45 may convert serial output received through the optical isolator 46 to a USB communication interface to facilitate coupling of output from the PIC controller 43 to the CPU 47. In one embodiment, the serial-to-USB converter 45 may be an FTDI model DLP-USB232M UART interface module. The converted USB signals may then be transmitted to the CPU 47 via a USB port for storage, printing, or display. The serial-to-USB converter 45 may also provide a +5VDC source that may be isolated by isolation DC/DC converter 49 for use by potentiostat 33 and other electronic components on the isolated side of the circuit.

The CPU 47 may be configured with software for displaying an analyte level in a desired graphical format on a display unit 50. The CPU 47 may be any commercial computer, such as a PC or other laptop or desktop computer running on a platform such as Windows, Unix or Linux. In one embodiment, the CPU 47 may be a ruggedized laptop computer. In another embodiment, the graphics displayed by the CPU 47 on the display unit 50 may show a numerical value representing real-time measurements, and also a historical trend, of the analyte of interest to best inform attendant health care professionals. The real-time measurements may be continuously or periodically updated. The historical trend may show changing analyte levels over time, for example, over one or more hours or days, for an analyte level such as blood glucose concentration.

The CPU 47 may provide power to the isolation DC/DC converter 49 and may also provide power to the display unit 50. The CPU 47 may receive power from a battery pack or a standard wall outlet (e.g. 120 VAC), and may include an internal AC/DC converter, battery charger, and similar power supply circuits. As shown in FIG. 3, the isolation DC/DC converter 49 may receive DC power from the CPU 47 via the bus labeled NON-ISOLATED PWS IN. In one embodiment, this DC power may be a +5VDC, 500 mA, +/−5% source provided, for example, via an RS232/USB converter (not shown). The +5VDC supply may be filtered at the non-isolated side of isolation DC/DC converter 47 using capacitors such as C37 and C38.

The isolation DC/DC converter 47 converts non-isolated +5VDC power to an isolated +5VDC source for output onto the bus labeled ISOLATED PWS OUT. In addition, the isolation DC/DC converter 47 may provide a physical isolation space for added immunity from electrical and magnetic noise. In one embodiment, the isolation space may be between about 0.3 inches and about 1.0 inches. In another embodiment, the isolation space may be 8 mm. The isolation DC/DC converter 47 may be a Transitronix model TVF05DO5K3 dual +/−5V output, 600 mA, regulated DC/DC converter with 6000 VDC isolation. The dual outputs +5V and −5V may be separated by a common terminal, and filtered using capacitors C33 and C36 between +5V and common, and capacitors C40 and C41 between −5V and common. Additional higher-order filtering may be provided to create multiple analog and digital 5V outputs, and to reduce any noise that may be generated on the isolated side of the circuit by digital switching of the components such as the ADC 41 and the PIC controller 43. For example, the +5V and −5V outputs may be filtered by inductors L1, L2, L3 and L4 configured with the capacitors C32, C35 and C39. In the configuration shown, these components provide a +5V isolated supply (+5VD) for digital components, a +/−5V isolated supply (+5VISO and −5VISO) for analog components, and an isolated signal ground for analog components.

In one embodiment, components of an analyte monitoring system may be mounted on one or more printed circuit boards contained within a box or Faraday cage. The components contained therein may include one or more potentiostats 33, R/V converters 37, low-pass filters 39, multiplexers 44, ADCs 41, PIC controllers 43, optical isolators 46, DC/DC converters 49, and associated isolated circuits and connectors. In another embodiment, the same board-mounted components may be housed within a chassis that may also contain serial-to-USB converter 45 and the CPU 47.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. An analyte monitoring system, comprising:
a biosensor sensing an analyte concentration in vivo and outputting a signal corresponding to the analyte concentration;
a controller receiving the signal and computing the analyte concentration therefrom; and
an isolation means coupled to the controller and isolating the biosensor from electrical and magnetic noise.

2. The analyte monitoring system of claim 1 further comprising a CPU coupled to the controller via the isolation means and receiving the computed analyte concentration for output to a display.

3. The analyte monitoring system of claim 2 further comprising an isolated circuit and a non-isolated circuit, the isolation means separating the isolated circuit from the non-isolated circuit, the isolated circuit including the biosensor and the controller, and the non-isolated circuit including the CPU.

4. The analyte monitoring system of claim 3 wherein the isolation means provides an isolation space between the isolated circuit and the non-isolated circuit between about 0.3 in. and about 1.0 in.

5. The analyte monitoring system of claim 4 wherein the isolation means comprises an optical isolator.

6. The analyte monitoring system of claim 4 wherein the isolation means comprises a DC/DC converter.

7. The analyte monitoring system of claim 2 further comprising a display unit coupled to the CPU, the isolation means electrically and magnetically isolating the display unit from the biosensor.

8. The analyte monitoring system of claim 7 wherein the CPU continually updates the computed analyte concentration for output to the display unit.

9. The analyte monitoring system of claim 1 wherein the biosensor comprises a four-electrode sensor.

10. The analyte monitoring system of claim 9 wherein the four-electrode sensor includes at least one enzyme electrode.

11. The analyte monitoring system of claim 10 wherein the enzyme electrode immobilizes glucose oxidase.

12. The analyte monitoring system of claim 3 further comprising a temperature sensor in the isolated circuit coupled to the controller.

13. The analyte monitoring system of claim 12 wherein the controller corrects the computed analyte concentration according to temperature sensed by the temperature sensor.

14. The analyte monitoring system of claim 13 wherein the sensed temperature represents temperature of the biosensor.

15. The analyte monitoring system of claim 12 further comprising a multiplexer in the isolated circuit, the multiplexer receiving the signal from the biosensor and a second signal from the temperature sensor and transmitting them as multiplexed output to the controller.

16. The analyte monitoring system of claim 15 further comprising an analog-to-digital converter in the isolated circuit, converting analog multiplexed output to digital data for the controller.

17. An isolated intravenous analyte monitoring system, comprising:
an isolated sensing circuit including
an in vivo biosensor sensing an analyte level in blood and outputting a signal corresponding to the analyte concentration; and
a controller receiving the digital data and computing therefrom a concentration of the analyte;
a non-isolated computer circuit including
a CPU processing the computed analyte concentration; and
a display unit coupled to the CPU and displaying the computed analyte concentration; and
an isolation device providing EMI isolation between the isolated sensing circuit and the non-isolated computer circuit.

18. The analyte monitoring system of claim 17 wherein the isolated sensing circuit further comprises:
a potentiostat converting output from the biosensor to voltage;
a multiplexer multiplexing voltage signals from the potentiostat; and
an analog-to-digital converter converting multiplexer output to digital data for output to the controller.

19. The analyte monitoring system of claim 18 wherein the isolated sensing circuit further comprises:
a thermistor sensing temperature of the amperometric sensor; and
a resistance-to-voltage converter converting resistance of the thermistor to voltage;
wherein the multiplexer multiplexes voltage output from the resistance-to-voltage converter with voltage output from the potentiostat.

20. The analyte monitoring system of claim 17 wherein the isolation device comprises a DC/DC converter.

21. The analyte monitoring system of claim 20 wherein the DC/DC converter receives non-isolated DC power from the CPU and provides isolated DC power to the isolated sensing circuit.

22. An isolated intravenous glucose monitoring system, comprising:
an in vivo enzyme electrode immobilizing glucose oxidase and outputting a signal proportional to blood glucose concentration;
an in vivo temperature sensor outputting a signal proportional to temperature of the enzyme electrode;
a controller computing a temperature-corrected blood glucose concentration signal from the temperature signal and the blood glucose concentration signal; and
an isolation means isolating the enzyme electrode, the temperature sensor, and the controller from electrical and magnetic noise.
